(19) United States
(12) Patent Application Publication
(10) Pub. No.: US 2025/0057399 A1
(43) Pub. Date: Feb. 20, 2025

(54) ENDOSCOPE WITH OFF-CENTRE HINGES FOR BENDING SECTION

(71) Applicant: AMBU A/S, Ballerup (DK)

(72) Inventors: Ken Henrik BUCH, Vordingborg (DK); Frederik Clausager Vemb HANSEN, Copenhagen NV (DK)

(73) Assignee: AMBU A/S, Ballerup (DK)

(21) Appl. No.: 18/805,749

(22) Filed: Aug. 15, 2024

(30) Foreign Application Priority Data
Aug. 18, 2023 (EP) 23192177.6

(51) Int. Cl. A61B 1/005; A61B 1/008

(52) U.S. Cl. CPC A61B 1/0052; A61B 1/008

(57) ABSTRACT

An endoscope including a bending section including segments forming a cable lumen portion for accommodation of an electrical cable, the cable lumen portion being offset from an endoscope axis and being arranged in a central plane substantially perpendicular to the bending plane, the segments connected by at least one first hinge device and at least one second hinge device, which define the first bending plane, with at least one of the segments being connected to a respectively adjacent one of the segments by a first hinge device defining a first hinge axis, which is offset from the central plane in a first direction and at least another one of the segments being connected to a respectively adjacent one of the segments by a second hinge device defining a second hinge axis, which is offset from the central plane in a second direction opposite to the first direction.

### CROSS-REFERENCE TO RELATED APPLICATIONS

This application claims priority from and the benefit of European Patent Application No. 23192177.6, filed Aug. 18, 2023; the disclosure of said application is incorporated by reference herein in its entirety.

### TECHNICAL FIELD

The present disclosure relates to endoscopes, and in particular to endoscopes comprising bending sections forming cable lumens.

### BACKGROUND

Steerable endoscopes often have a proximal handle and a distal insertion cord including a bending section and a distal tip. To deflect the distal tip, the bending section can be bent or manipulated by pulling one or more pulling/steering wires, which extend into the insertion cord of the endoscope and which have distal portions attached to the bending section. The bending section usually has segments, which are connected via hinges.

Typically, in an endoscope which allows two-way-bending, two pulling wires may be provided, which are circumferentially offset with respect to each other by 180° and pulling either one of the pulling wires/steering wires will result in a bending motion in a corresponding direction. Thus, one degree of freedom (one bending plane) is provided comprising an up/down movement or a left/right movement of the bending section effected by pulling one of two diametrically opposed pulling wires. Proximally, the two pulling wires are often connected to one control wheel to operate the pulling wires.

Similarly, endoscopes which allow four-way-bending generally comprise four pulling wires circumferentially offset by 90°. In this case, one pair of diametrically opposed pulling wires operates the up/down movement of the bending section (i.e. in a first bending plane/providing a first degree of freedom) e.g. via one control wheel. The other two diametrically opposed pulling wires may operate the left/right movement of the bending section (i.e. in a second bending plane/providing a second degree of freedom), e.g. via another control wheel.

Insertion cords of endoscopes usually have diameters which are as small as possible and/or which provide room for large working channels so that relatively large instruments may be inserted through said working channels. In particular for endoscope insertion cords with a small outer diameter, there is not enough space in the bending section to have hinges and electrical cables (in short: cable) in the same plane. If the cable is displaced from a center plane, the bending of the bending section will result in an interchanging pull and push on the cable, said pull and push resulting in bulges and potential damage to the cable.

### BRIEF DESCRIPTION OF THE DISCLOSURE

In view of the above-described problems, it is an object of the present disclosure to provide an endoscope which shall reduce or avoid the disadvantages of the related art. In particular, it is an object of the disclosure to provide a steerable endoscope with an insertion cord having a bending section with a relatively large working channel relative to its outer diameter and which reduces strain on electrical cables.

In a first aspect, the present disclosure provides an endoscope. In a second aspect, the present disclosure provides a visualization system including the endoscope and a monitor.

In an embodiment according to the first aspect, the endoscope comprises a bending section that comprises segments connected by first and second hinges in a one-piece part. The first hinges and the second hinges are disposed in an alternating manner on opposite sides of a central plane along a length of the bending section. Thusly, the first hinges connect a pair of adjacent segments, the second hinges connect a second pair of adjacent segments, another pair of first hinges connects a third pair of adjacent segments, and so on. This allows for a balanced bending of the bending section and reduces irregularities (e.g. a corrugated or wave-like plane) in the bending plane and the neutral plane during bending of the bending section. Thus, bulging and potential damage of the electrical cable is further reduced. That is, the hinge devices may form a spine meandering from one side of the central plane to another side of the central plane via the hinge devices (i.e. first, second hinge devices).

The meandering spine allows a cable lumen to be positioned closer to the outer surface of the bending section, thereby freeing space to increase the size of the working channel. Additionally, placing the cable lumen close to the outer surface allows the first hinges and the second hinges to lie co-extensively, at least in part, with surfaces, or faces, of the cable lumen, to thereby ease threading of the cable through the cable lumen and support the cable passing therethrough.

In one example, a first segment is connected to a second segment by a first pair of first hinges, the second segment is connected to a third segment by a first pair of second hinges, the third segment is connected to a fourth segment by another pair of first hinges, the fourth segment is connected to a fifth segment by another pair of second hinges and so on, where the first, second, third, fourth, and fifth segments are disposed sequentially along the length of the bending section.

The segments may be aligned along a bending section longitudinal axis and form a cable lumen portion for accommodation of at least one electrical cable, said cable lumen portion being offset from the endoscope axis and arranged in a cable, or central, plane, which is substantially perpendicular to a first bending plane. The first hinges form a first hinge device and the second hinges form a second hinge device, which define the first bending plane and are configured to allow bending of the bending section in the first bending plane, with at least one of the segments being connected to a respectively adjacent one of the segments by a first hinge device, defining a first hinge axis, which is offset from the central plane in a first direction, and at least another one of the segments being connected to a respectively adjacent one of the segments by a second hinge device, defining a second hinge axis, which is offset from the central plane in a second direction opposite to the first direction.

The endoscope may comprise a proximal handle or interface; an insertion cord extending along an endoscope axis from the handle or interface and configured to be inserted into a patient's body cavity, the insertion cord comprising an insertion tube, the bending section (described above), and a distal tip; and a steering mechanism configured to bend the bending section at least in the first bending plane. Such bending may occur intra-operatively while the endoscope tip is navigated inside a patient.

Expressed in other words, corresponding hinge devices comprise at least one first hinge device and at least one second hinge device, which are offset from each other with a central plane arranged between them. When the bending section is bent in the first bending plane, the segments are tilted relative to each other via the at least one first and second hinge devices (i.e. the at least one first hinge device and the at least one second hinge device). The first hinge device connects at least one pair of the segments and the second hinge device connects another pair of the segments, i.e. not the same pair of segments as the first hinge device. That is, two adjacent ones of the segments are connected either by the first hinge device or by the second hinge device and not by the first and the second hinge device.

The pairs of the segments may be two separate or overlapping pairs of the segments. I.e. in separate pairs of the segments, one pair may include two segments that are different from two other segments forming another pair. E.g., one pair may be formed by first and second ones of the segments and another pair may be formed by third and fourth ones of the segments.

In the present embodiment, adjacent ones of the segments are respectively connected via hinge devices (first and second hinge devices), with some of the hinge devices being arranged selectively on one side of the central plane, i.e. to one side of the cable lumen portion, and some of the hinge devices being arranged on another side of the central plane/the cable lumen portion. The first and second hinge devices are arranged alternatingly along the endoscope axis, i.e. the segments are alternatingly connected by first hinge devices and second hinge devices. That is, two of the segments are connected via one of the first hinge devices and the next two segments are connected via one of the second hinge devices, and so forth.

The segments may be separated by gaps (e.g slots or slits), formed by laser cutting, cutting or molding, with at least a first one of the gaps being bridged by a first hinge device and at least a second one of the gaps, which is axially offset/displaced from the first one of the gaps, being bridged by a second hinge device. The first hinge device and the second hinge device may be circumferentially offset from each other on opposing sides of the central plane. Preferably, the gaps extend in a plane substantially orthogonal to the endoscope axis. Optionally, at least one web-like wall structure may be left between adjacent ones of the segments to form the first hinge device or the second hinge device.

In one variation of the present embodiment according to the first aspect, the two pairs of segments (each pair connected by first or second hinge devices) may be connected via e.g. a third or fourth hinge device for four-way bending. In overlapping pairs, an intermediate one of the segments may form a pair with a distally adjacent one of the segments and may further form another pair with a proximally adjacent one of the segments. E.g., one pair may be formed by first and second ones of the segments and another pair may be formed by second and third ones of the segments.

The central plane may be a plane which coincides with or extends near a first neutral plane, which is substantially neutral with regard to a bending of the bending section in the first bending plane. “Near” in this case means that the central plane and the neutral plane are offset from each other by a maximum offset of 50%, preferably 30%, further preferably 10%, of a diameter of the bending section. Most preferably, the central plane is the same as the first neutral plane. The first neutral plane is determined as follows: When the bending section is bent in the first bending plane, a length of the bending section on an inner curvature side is shortened. Further, a length of the bending section on an outer curvature side is lengthened. Intermediately, perpendicular to the first bending plane and preferable in a center of the bending section, there is a plane in which a length of the bending section remains the same in the neutral state and in a bent state of the bending section. Said plane is called the neutral plane. The neutral plane may be defined by the first and second hinge devices, in particular arranged between the first and second hinge devices, preferably centrally between them. In particular, the first hinge axis may be defined by parameters such as a position of the first hinge axis at the bending section and by a bending stiffness, e.g. defined by a shape and thickness of material, of the first hinge device. The second hinge axis may be similarly defined by the second hinge device. Depending on said parameters, the first and/or second hinge axis may be inclined or parallel with respect to each other and to the neutral plane or central plane. E.g., the first and second hinge axis may extend in a V-shape with respect to each other. Together, the first and second hinge axis of the at least one first and second hinge devices may define the neutral plane and/or central plane.

The endoscope is preferably a single-use endoscope. In the insertion cord, the insertion tube, the bending section and the distal tip are arranged in this order starting from the proximal handle or interface. The endoscope axis is a central axis of the insertion cord, with the insertion cord extending along said endoscope axis. Expressions such as “axially”, “radially” and “circumferentially” relate to directions in relation to said endoscope axis. The bending plane is a plane extending along the endoscope axis and staying constant during a bending action. I.e. when the bending section is bent in the first bending plane, it moves within the bending plane and the distal tip is moved in the bending plane/moves within the bending plane, without the bending plane itself being bent. In other words, the distal tip is moved in a first or an opposing second direction by bending the bending section in the first bending plane, with the first and the second direction coinciding with/extending in the first bending plane.

Two elements being offset from each other means that they are displaced from one another by an angular and/or linear displacement or distance, said angular and/or linear displacement or distance being larger than zero. The first hinge axis and the second hinge axis may be offset from the central plane by a predetermined distance or angle. A direction of the offset of the first hinge axis with respect to the central plane is opposite to a direction of the offset of the second hinge axis. The first and second hinge devices (i.e. the first hinge device and the second hinge device) are preferably arranged symmetrically with respect to the central plane. The first and second hinge devices allow tilting of the correspondingly adjacent segments with respect to each other around the first hinge axis or respectively the second hinge axis. In particular, the first and second hinge devices are provided only for bending the bending section in the first bending plane, not in additional bending planes. A bending of the bending section in other bending planes may be substantially independent from the first and second hinge devices and may be provided via additional hinge devices, as discussed in more detail further below.

The endoscope comprises the electrical cable extending through the segments of the bending section. The electrical cable is, preferably, a cable supplying electrical power and/or control signals to the distal tip, particularly to an optical unit/camera module arranged at the distal tip.

The optical unit may be a camera chip and/or illumination means. I.e. the electrical cable may comprise one or more wires for transfer of signals such as sensory data and/or control commands and/or electrical energy.

The steering mechanism may include at least one steering wire, preferably two steering wires for bending of the bending section in the first bending plane. A distal end of the at least one steering wire is preferably connected at a distal end portion of the endoscope, particularly a distal end of the bending section. Further, the steering mechanism preferably comprises a control wheel arranged in or at the handle and being connected to a proximal end of the at least one steering wire to drive the bending of the bending section in the first bending plane via the at least one steering wire. The control wheel is an example of a steering actuator. Other steering actuators, such as lever and sliders, which are well known in the art, may also be used.

Advantageously, the endoscope as described above will allow placing of the electrical cable in or near a plane that is substantially neutral with respect to a bending action of the bending section in the first bending plane. Thus, a lengthening or compressing of the electrical cable due to the bending will be reduced, preferably minimized. In detail, only very small and local lengthening and compressing between the first and second hinge devices due to the alternatingly offset position of said first and second hinge devices will be applied to the electrical cable. Accordingly, a risk for damaging the electrical cable will advantageously be reduced.

Preferably, the first hinge device, particularly the first hinge axis, is offset from the central plane by less than half a diameter of the bending section, preferably by a maximum of 40%, further preferably by a maximum of 30%, further preferably by a maximum of 20% of the diameter of the bending section. Further preferably, the second hinge device, particularly the second hinge axis, is offset from the central plane by less than half a diameter of the bending section, preferably by a maximum of 40%, further preferably by a maximum of 30%, further preferably by a maximum of 20% of the diameter of the bending section. The offset mentioned above is a maximum distance of the first or respectively second hinge device, particularly the first or second hinge axis, with respect to the central plane in a direction perpendicular to the central plane. Alternatively, each of the at least one first hinge device, particularly the first hinge axis, and the at least one second hinge device, particularly the second hinge axis, is at an angular position offset from the central plane by less than 90°, preferably by a maximum of 60°, further preferably a maximum of 45°, further preferably a maximum of 30°, further preferably a maximum of 20°.

In particular, it is preferred that the first and/or second hinge devices are formed directly adjacent to the cable lumen portion, preferably such that the first and/or second hinge devices form part of a cable lumen inner surface. Thus, irregularities in an extension of the neutral plane or central plane can be minimized particularly well. Additionally and synergistically, a good support of the electrical cable in the cable lumen portion may be achieved even in an area between the segments.

Further, the first hinge device and/or the second hinge device are formed as film/strip hinges. In particular, the first and second film hinges are formed in one piece of material together with the segments, i.e. the segments of the bending section and the film hinges, preferably all of them, are formed in one piece of material. In particular, the first hinge device and/or the second hinge device are formed as strips of material bridging the gaps between the respectively adjacent segments. This allows a particularly space-saving construction and particularly cost-efficient manufacturing and assembly of the endoscope.

The cable lumen portion is preferably offset from the endoscope axis and, further preferably, a central axis of the cable lumen portion extends parallel to the endoscope axis. In particular, the cable lumen portion is arranged in a radially outer area of the bending section. Preferably, only a thin wall portion of the segments (referred to below as “upper wall portion”) separates the cable lumen portion from a radially outer surface of the segments, i.e. no other elements are received in the segments radially outside of the cable lumen portion. Thus, the endoscope will allow maximizing of a radial distance of the electrical cable with respect to the endoscope axis, thus leaving more room for a working channel accommodated in the bending section. This makes it possible to advantageously provide a large working channel even with small endoscopes and allows insertion of relatively large instruments through said working channel. In particular, the endoscope may comprise a working channel extending through the segments of the bending section.

Preferably, a wall thickness of the segments in or near the central plane, particularly in the circumferential area of the cable lumen portion, further particularly adjacent to the cable lumen portion on a radially outer side thereof, is thinner than a thickness of a hinge element of the at least one first hinge device and/or the at least one second hinge device. Expressed in other words, the cable lumen portion may be arranged between the first and second hinge devices directly in the circumferential direction. I.e. the cable lumen portion may overlap with the first and second hinge devices when viewed along the circumferential direction. This is possible, since very little stress is applied to the electrical cable and the segments in the area of the neutral plane or central plane. Thus, material cost can advantageously be reduced and an available space for a working channel may be increased further.

Preferably, each of the at least one first hinge device and/or the at least one second hinge device comprises two hinge elements, with one of said hinge elements being arranged on one side of the first bending plane and the other one of the hinge elements being arranged on the other side of the first bending plane, preferably such that the two hinge elements are arranged mirror symmetrically with respect to the first bending plane. Expressed in other words, each of the one or more first hinge devices may have two hinge elements connecting adjacent ones of the segments and/or each of the one or more second hinge devices may have two hinge elements connecting adjacent ones of the segments. The first hinge axis may extend between the hinge elements of the first hinge device. The second hinge axis may extend between the hinge elements of the second hinge device. Preferably, the hinge elements are arranged such that the first hinge axis and/or the second hinge axis extend parallel to the central plane and/or the neutral plane. Thus, the bending section may have a particularly simple structure, which is simple and cost-efficient in manufacture.

Between each adjacent ones of the segments, two radially opposite gaps are provided, which separate the segments from each other, with the first or second hinge device, in particular the respective hinge elements, separating the radially opposite gaps from each other. In particular, circumferential ends of each two radially opposite gaps face each other. The radially opposite gaps form circumferential ends. The gaps will not overlap in the axial direction and do not have portions extending circumferentially next to each other. Preferably, one of the radially opposite gaps extends further through the bending section than the longitudinal axis to provide an offset of the first or second hinge devices or the respective hinge elements from the central plane or neutral plane.

The segments form a major lumen portion, which has a larger cross-sectional area than the cable lumen portion. The cable lumen portion and the major lumen portion together may provide a substantially pear-shaped lumen without an additional wall portion therebetween. Thus, more space for accommodation for a working channel tube is available and a larger working channel tube may be used. However, an embodiment where the major lumen portion and the cable lumen portion are separated by a wall portion could also be provided. That is, a solution where the cable lumen is closed off from the major lumen portion to avoid slipping of the cable on a working channel surface can be provided.

In a transitional lumen wall area connecting the cable lumen portion and the major lumen portion, a lumen wall is formed convexly. The transitional lumen wall area bulges or protrudes radially inwards with respect to the longitudinal axis. I.e. the transitional lumen wall extends further inwards than a line being tangential with respect to the cable lumen portion and the major lumen portion. The transitional lumen wall area may be a wall portion formed by the segment in an area where the cable lumen portion and the cable lumen portion are merged. The transitional wall area provides lateral support for the electrical cable. Due to this, the electrical cable may be supported on a radially inwards facing side by the working channel and adjacent to said working channel, particularly on all other sides, by a wall formed by the segments. Advantageously, a risk that the electrical cable may slip into the major lumen portion between a wall of said major lumen portion and the working channel accommodated therein may be minimized and a support for the electrical cable may be optimized.

Preferably, the major lumen portion is arranged in a cross-sectional area comprising the longitudinal axis. In particular, the major lumen extends from the cable lumen portion to a diametrically opposite radially outer wall of the segments. Thus, a size of the major lumen may be optimized and a larger working channel may be used. Further preferably, the major lumen portion extends along an axis extending in the central plane, preferably offset from the longitudinal axis. Therefore, a size of the major lumen portion may be further increased.

Preferably, the segments further form at least one steering wire lumen portion for accommodation of at least one steering wire, said steering wire lumen portion being arranged at one side of the central plane, i.e. offset from the central plane. Due to this, efficient force transmission via the steering wires for bending the bending section is possible. Preferably, the at least one steering wire lumen portion is arranged in or near the first bending plane, i.e. offset from the first bending plane at a maximum angle of 45°, further preferably 30°, further preferably 10°. Two such steering wire lumen portions may be arranged on opposing sides of the central plane, preferably mirror symmetrically, with respect to the central plane. Moreover, the at least one steering wire lumen portion may preferably be laterally connected to the major lumen portion and/or to the cable lumen portion. In particular, the at least one steering wire lumen portion may be directly connected to the major lumen portion, i.e. not via the cable lumen portion or other lumen portions. Advantageously, this allows further increasing of the major lumen portion. Alternatively the at least one steering wire lumen may be closed off from the major lumen and/or the cable lumen portion, i.e. may be separated via a wall portion. Alternatively, a gap separating the steering wire lumen portion from the major lumen portion is sufficiently narrow to prevent lateral movement of the steering wire.

The endoscope may be a two-way bending endoscope, which has only one bending plane. The two-way bending endoscope allows realizing the advantages of the present application particularly well, particularly with regard to reducing a risk of damaging the electrical cable during bending of the bending section.

The endoscope may be a four-way bending endoscope. That is, the endoscope has exactly two bending planes which are, preferably, orthogonal to each other. The central plane may be parallel to the second bending plane. The second bending plane may be defined by third hinge devices connecting a number of the segments. The third hinge devices may be arranged in the first bending plane for a particularly simple design. Alternatively, the second bending plane may be defined by the third hinge devices and by fourth hinge devices connecting respectively adjacent segments, the third and fourth hinge devices being alternatingly offset with respect to the first bending plane in a manner similar to that described above with regard to the first and second hinge devices and their offset from the central plane.

Preferably, the first and second hinge devices and the third, and optionally fourth, hinge devices may be arranged alternatingly. In other words, along the axial direction (i.e. along the longitudinal axis), a first one of the segments may be connected to a subsequent second one of the segments via one hinge device for bending in the first bending plane (i.e. one of the first and second hinge devices) and the second one of the segments may be connected to a subsequent third one of the segments via one hinge device for bending in the second bending plane (i.e. one of the third and optionally fourth hinge devices), and the third one of the segments may be connected to a subsequent fourth one of the segments via another hinge device for bending in the first bending plane (i.e. the other one of the first and second hinge devices), and the fourth one of the segments may be connected to a subsequent fifth one of the segments via another hinge device for bending in the second bending plane (i.e. another third hinge device or the other one of the third and fourth hinge devices). For example, along the axial direction, a first hinge device may be followed by a third hinge device, which may be followed by a second hinge device, which may be followed by another third or a fourth hinge device, and then again a first hinge device, and (optionally) so on.

For the sake of better clarity, optionally, the first hinge device may be called a first plane-one hinge device (i.e. a first hinge device for bending the bending section in the first bending plane) and the second hinge device may be called a second plane-one hinge device (i.e. another hinge device for bending the bending section in the first bending plane). Further, for the sake of clarity, the third hinge device may be called a first plane-two hinge device (i.e. a first hinge device for bending the bending section in the second bending plane). Yet further, in case there are third and fourth hinge devices as discussed above, the fourth hinge device may be called a second plane-two hinge device (i.e. another hinge device for bending the bending section in the second bending plane).

Preferably, the electrical cable is fixed at the distal tip and/or at a distal end portion of the bending section. Further preferably, the electrical cable is loosely held in the bending section, preferably in the entire bending section. In other words, the electrical cable is preferably not fixed to the cable lumen portion of the segments of the bending section. This advantageously allows axial shifting of the electrical cable at least within the bending section. In this manner, the shifting of the electrical cable may balance out minimal lengthening and compressing, which may be applied to the electrical cable between each two segments during bending of the bending section due to the offset of the first and second hinge devices from the central plane.

Further preferably, at a distal end segment of the segments, i.e. at a most distal one of the segments, a steering cable is held or anchored. In particular, the most distal one of the segments may form a wire holder or wire attachment portion for holding or attaching the at least one steering wire. Said wire holder may simply provide a distally facing stop surface for a stopper element connected to a distal end of the respective steering wire. This provides a simple design of the bending section, facilitates assembly of the endoscope and allows a direct and efficient appliance of operational force for bending the bending section. The most distal one of the segments may further form a mounting portion for connection of the distal tip. Further, the most proximal one of the segments may form a mounting portion for connection of the insertion tube.

### BRIEF DESCRIPTION OF THE DRAWINGS

The following figures illustrate some embodiments of the disclosure. The disclosure is not limited to the embodiments described below. Other embodiments, combinations of embodiments, and modifications thereof, are within the scope of protection defined by the claims.

FIG. 1 shows a schematic view of a visualization system comprising an endoscope and a monitor according to the present disclosure.

FIG. 2 shows a perspective view of a proximal end of a bending section according to the present disclosure.

FIG. 3 shows a cross-sectional view through the bending section of FIG. 2 along a first hinge device.

FIG. 4 shows a cross-sectional view through the bending section of FIG. 2 along a second hinge device.

FIG. 5 shows a frontal view of a most distal segment of the bending section of FIG. 2.

FIG. 6 shows a side view of the bending section of FIG. 2 orthogonal to a first bending plane B.

FIG. 7 shows a side view of the bending section of FIG. 2 orthogonal to a central plane C.

FIG. 8 is a a cross-sectional view of the bending section of FIGS. 2-7 and 9-13.

FIG. 9 shows a cross-sectional view of the bending section of FIGS. 2-5 along a central plane.

FIG. 10 shows a perspective sectional view of the bending section of FIGS. 2-5 cut along the central plane.

FIG. 11 shows a simplified perspective view of a variation of the bending section of FIG. 2.

FIG. 12 shows a cross-sectional view of the bending section of FIG. 11 in an area of a third hinge device.

FIG. 13 is a a cross-sectional view of the bending section of FIGS. 11 and 12 along the central plane.

### DETAILED DESCRIPTION

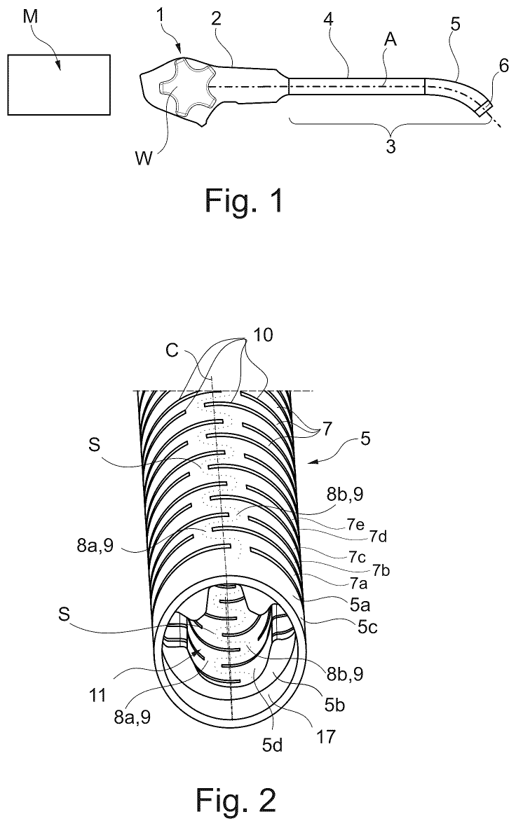

FIG. 1 shows a schematic view of an endoscope 1, which is preferably a single-use endoscope. The endoscope 1 has a handle 2 and an insertion cord 3 extending distally from the handle 2 along an endoscope axis A concentric with a bending section longitudinal axis L. The insertion cord 3 has an insertion tube 4 connected to the handle 2, a bending section 5 connected to a distal end of the insertion tube 4, and a distal tip 6 connected to a distal end of the bending section 5. At the distal tip 6, image capturing means, such as a miniature video camera, and illuminating means, such as light-emitting diodes or optical fibres connected to a proximal source of light, are arranged/installed, such that the patient's body cavity can be illuminated and inspected. An image captured by the image capturing means can be shown on a monitor M. The monitor M is provided separately from and connectable, wired or wirelessly with the endoscope 1.

FIG. 2 shows a perspective view of a proximal portion of the bending section 5 of an embodiment of the present disclosure. The bending section 5 comprises a tubular wall 5a, which forms a number of substantially ring-shaped segments 7. Said segments 7 are aligned along the longitudinal axis L. The segments 7 are connected via hinge devices 8a, 8b. The tubular wall, including the segments 7 and the hinge devices 8a, 8b, is formed in a one-piece part. Each of the hinge devices 8a, 8b comprises two hinges, or hinge elements, 9. Other than at the hinge devices 8a, 8b, the segments 7 are separated by gaps, or slits, 10 extending substantially perpendicular to the longitudinal axis L. Further, the tubular wall 5a comprises a thickness that varies, forming transverse portions 5b on at least some of the segments, and the transverse portions 5b have cross-sections defining a lumen 11 extending along the longitudinal axis L. The transverse portions 5b extend from an outer surface 5c to an inner surface 5d of the tubular wall 5a. The lumen 11 is formed by the inner surface 5d of the tubular wall 5a. The lumen 11 comprises a major lumen portion 11a, a cable lumen portion, or lobe, 11b, and steering wire lumen portions, or lobes, 11c. The lumen 11 has a transverse periphery that includes the peripheries of the major lumen portion 11a and the lobes 11b-c. The major lumen portion 11a has a substantially circular shape and the lobes 11b-c extend outwardly from the major lumen portion 11a. The lumen 11 is formed by lumens of the segments, each of the lumens of the segments including a major lumen portion 11a and the lobes 11b-c. Thus, parts 11 and 11a-c refer to the respective lumen/lumen portion of the bending section in aggregate and of each segment forming the overall lumen/lumen portion. Some segments may omit at least one of the lumen portions.

The bending section 5 has a meandering spine S extending along the longitudinal axis L. A central plane C traverses the meandering spine S. The first hinge devices 8a comprise first hinges and the second hinge devices 8b comprise second hinges. The first hinges and the second hinges are disposed in an alternating manner on opposite sides of the central plane C along a length of the bending section 5. The first hinges connect a pair of adjacent segments, the second hinges connect a second pair of adjacent segments, another pair of first hinges connects a third pair of adjacent segments, and so on, thus defining the meandering spine S, where the first hinges are transversely offset from, and thus do not longitudinally overlap, the second hinges.

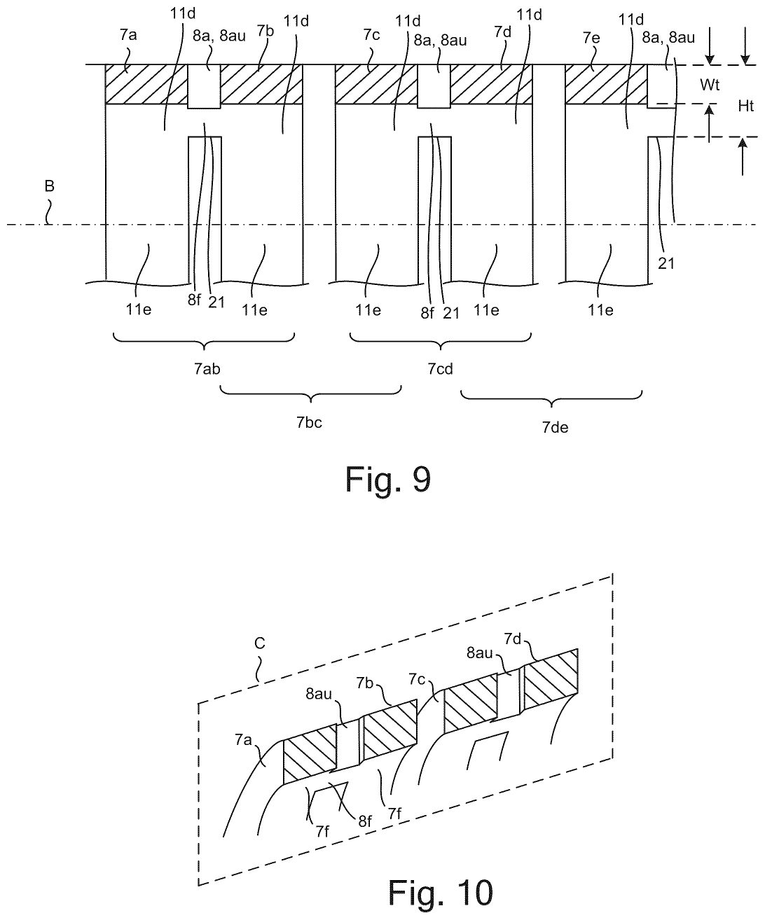

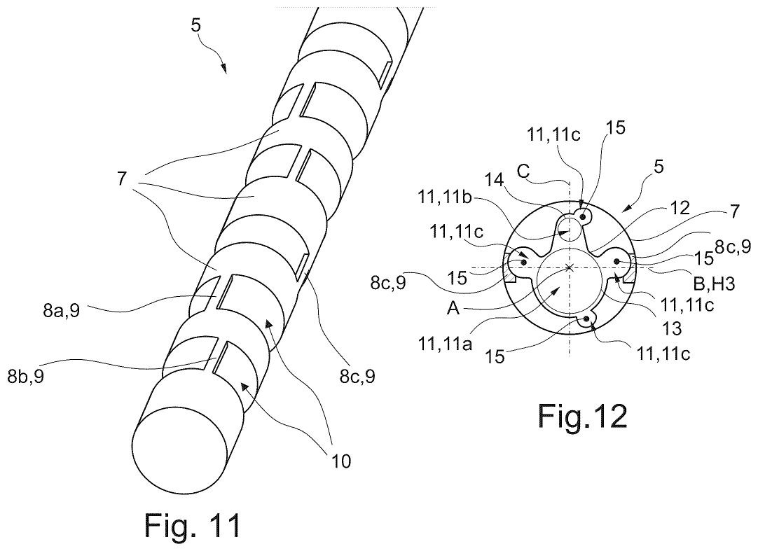

Segments 7a-7e are identified to exemplify the foregoing. A first segment 7a is connected to a second segment 7b by a first pair of first hinges (first hinge device 8a), the second segment 7b is connected to a third segment 7c by a first pair of second hinges (second hinge device 8b), the third segment 7c is connected to a fourth segment 7d by another pair of first hinges, the fourth segment 7d is connected to a fifth segment 7e by another pair of second hinges, where the first, second, third, fourth, and fifth segments are disposed sequentially along the length of the bending section. The first and second segments comprise a first pair of adjacent segments, the second and third segments comprise a second pair of adjacent segments, the third and fourth segments comprise a third pair of adjacent segments, and the fourth and fifth segments comprise a fourth pair of adjacent segments (see FIG. 9). In this example, all the pairs of adjacent segments are also adjacent. However, in a variation of the present embodiment described with reference to FIGS. 11-13, the pairs of segments are separated by segments connected by third, and optionally fourth, hinge devices, providing a four-way bending section.

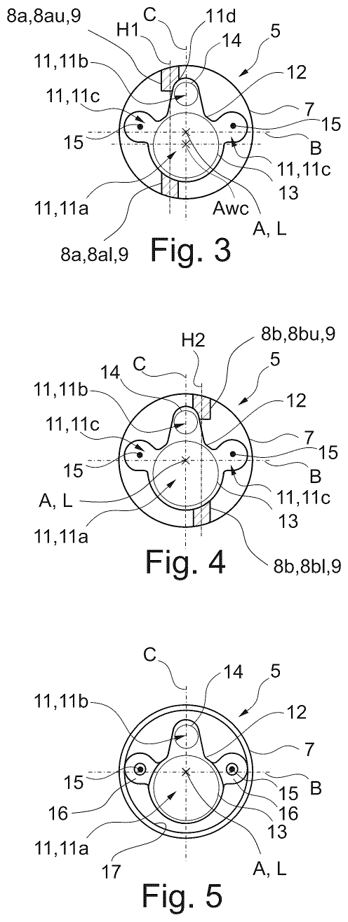

FIGS. 3-10 will describe an endoscope comprising:
a proximal handle or interface;
an insertion cord extending distally from the handle or interface, the insertion cord comprising an insertion tube, a bending section and a distal tip;
a working channel tube; and
an electrical cable extending from the handle and through the bending section,
the bending section comprising a one-piece part including a longitudinal axis, segments, first hinge devices, and second hinge devices, the first hinge devices and the second hinge devices being configured to allow bending of the bending section in a bending plane, each of the first hinge devices comprising a pair of hinges and a first hinge axis, and each of the second hinge devices comprising a pair of hinges and a second hinge axis, the first hinge axes being parallel and transversely offset from the second hinge axes,
each of the segments comprising a wall with a varying thickness between an outer surface and an inner surface thereof, the inner surface forming a lumen including a major lumen portion, a cable lumen lobe, and opposite steering wire lumen lobes, the major lumen portion receiving the working channel tube therethrough,
wherein the segments are alternatingly connected to each other by the first hinge devices and the second hinge devices, forming at least one meandering spine,
wherein the cable lumen lobe is offset from the longitudinal axis, a central plane traversing the cable lumen portion and the longitudinal axis, the central plane being positioned between the first hinge axes and the second hinge axes.

In one variation, the the central axis is perpendicular to the bending plane and parallel with the first hinge axes and the second hinge axes, and each of the pair of hinges of the first hinge devices and the second hinge devices includes an upper hinge adjacent to a respective one of the cable lumen lobes.

In another variation, each of the upper hinges comprises an inner upper hinge face, wherein each of the cable lumen lobes comprises a first face opposite a second face, the first face and the second face being part of the lumen, wherein each of the inner upper hinge faces of the first hinge devices has a common edge, and extends co-extensively, with a respective one of the first faces, and wherein each of the inner upper hinge faces of the second hinge devices has a common edge, and extends co-extensively, with a respective one of the second faces.

In a further variation, each segment comprises an upper wall portion of the wall, the upper wall portion traversed by the central plane and adjacent to the cable lumen lobe, the upper wall portion being thinner than a thickness of the upper hinges.

In yet another variation, the first hinge device is offset from the central plane by at most 20% of an outer diameter of the bending section, and the second hinge device is offset from the central plane by at most 20% of the outer diameter of the bending section.

In a yet further variation, the major lumen portion comprises arcuate portions supporting the working channel tube, whose lengths, in aggregate, are larger than 50% of a circumferential length of the working channel tube, the bending section has an outer diameter smaller than 3.2 mm, and an internal diameter of the working channel tube is at least 45% of the outer diameter of the bending section.

FIGS. 3-5 show cross-sectional views of the bending section 5. FIG. 3 shows a cross-section through the first hinge device, FIG. 4 shows a cross-section through the second hinge device, and FIG. 5 shows a frontal view of a most distal segment. These figures show a first bending plane B which is substantially perpendicular to the central plane C, which intersects said first bending plane B along the longitudinal axis L, a first hinge axis H1 traversing the first hinge device 8a, and a second hinge axis H2 traversing the second hinge device 8c. The first hinge axis H1 and the second hinge axis H2 are parallel with, and on opposite sides of, the central plane C. The lower hinges support the working channel tube in the same manner that the upper hinges support the cable. Similarly, the lower hinges may co-extend with opposing faces of the main lumen 11a.

As shown, the first hinge device 8a comprises an upper hinge 8au and a lower hinge 8al, and the second hinge device 8b comprises an upper hinge 8bu and a lower hinge 8bl. The upper and lower hinges are axially aligned, on axis H1 or H2. However, in a variation of the present embodiment, the lower hinges are offset a different amount than the upper hinges. For example, the lower hinges may be closer transversely to each other than the upper hinges. In another variation, the lower hinges do not alternate and instead lie on a common plane, which may be the central plane C. Such lower hinges may increase flexibility of the bending section while the upper hinges still provide the support for the cable.

The bending section 5 is configured to bend in at least two different directions in at least a first bending plane B. This enables steering of the endoscope 1 with a steering mechanism described in more detail below. In a neutral state, the bending section 5 is unbent by the steering mechanism, preferably straight/in alignment with the insertion tube 4. When the bending section 5 is bent in the first bending plane B, a length of the bending section 5 on an inner curvature side is shortened due to compression of the gaps 10. Further, a length of the bending section 5 on an outer curvature side is lengthened due to expansion of the gaps 10. Intermediately, there is a plane in which a length of the bending section 5 remains the same in the neutral state and in a bent state. Said plane is the neutral and/or central plane C. Preferably, the neutral and/or central plane C may be orthogonal to the first bending plane B.

The hinge devices 8a, 8b include a number of first hinge devices 8a and second hinge devices 8b. Each hinge element 9 is formed by a connecting web, or strip, of material extending between and connecting adjacent ones of the segments 7. The bending section 5 may be injection molded in one-piece of polymer material. The polymer material strips can bend. The molds of the injection molding machine can comprise a mandrel and two semi-circular elongate mold parts that come together around the mandrel and form the segments, the hinges, and the gaps therebetween.

The segments 7 are formed by the tubular wall forming a lumen 11. The lumen 11 forms a continuous hollow with several lumen portions. Said lumen portions include a major lumen portion 11a, which is the largest among the lumen portions. The major lumen portion 11a e.g. forms or is configured to accommodate a working channel tube 13 that can be used to insert surgical instruments, flushing or the like. The working channel tube 13 is schematically shown in FIGS. 3-5. A central axis Awc of the major lumen portion 11a may lie in the central plane C. A cross-sectional area of the major lumen portion 11a comprises the longitudinal axis L. Further, the central axis Awc of the major lumen portion 11a is offset from the longitudinal axis L, such that the major lumen portion is arranged opposite the cable lumen portion 11b.

The cable lumen portion 11b accommodates the electrical cable 14, which comprises wires extending to an image sensor of the camera to transmit control signals, image signals, and power. The cable lumen portion 11b is preferably connected to the major lumen portion 11a as shown in this embodiment and has a central axis that may lie in the central plane C. The central axis may coincide with a central axis of the electrical cable 14. Further, the central axis of the cable lumen portion 11b is offset from the longitudinal axis L to a side opposite of the central axis Awc of the major lumen portion 11a. A cross-sectional area, and a diameter, of the cable lumen portion 11b is smaller than that of the major lumen portion 11a. Transitional lumen wall areas 12 (of the inner surface of the wall) connect the cable lumen portion 11b and the steering wire lumen portions 11c on each side. The steering wire lumen portions 11c may be directly connected (as shown) to the major lumen portion 11a or may be connected via transitional lumen wall areas. The steering wire lumen portions 11c accommodate corresponding steering wires 15, which may be enclosed in wire sheaths.

The steering wires 15 are part of the steering mechanism for bending of the bending section 5. A proximal end of the steering wires 15 is connected to the control wheel W arranged in or at the handle 2 for operating the steering wires 15. The steering wire lumen portions 11c are preferably connected to the major lumen portion 11a and the cable lumen portion 11b on opposing sides with respect to the central plane C, as shown in the present embodiment. In particular, one of the steering wire lumen portions 11c is arranged at or near one of the transitional areas 12 and the other one of the steering wire lumen portions 11c is arranged at or near the other one of the transitional areas 12. Further, central axes of the steering wire lumen portions 11c lie in or near the first bending plane B. A cross-sectional area or diameter of each steering wire lumen portion 11c is smaller than that of the major lumen portion 11a. Although a wheel W is shown, a bending mechanism may comprise, instead, a lever connected to an axle located inside the handle which is connected to the steering wires, as is known in the art.

FIG. 3 further shows the first hinge device 8a. Said first hinge device 8a is formed by two hinges, or hinge elements, 9. The hinge elements 9 of the first hinge device 8a are arranged on opposite sides of the first bending plane B, particularly at mirror symmetrical locations with respect to the first bending plane B. Further, the hinge elements 9 of the first hinge device 8a are offset from the neutral or center plane C to one side, i.e. both to the same side. The first hinge axis H1 traverses the first hinge device 8a, particularly its hinge elements 9. The location of the first hinge axis H1 is defined by the location of the first hinge device 8a. In the present embodiment, said first hinge axis H1 is parallel to the central plane C.

As shown in FIG. 3 (and FIG. 4), the first (and/or second) hinge devices are formed directly adjacent to the cable lumen portion 11b so that the hinge devices extend a cable lumen inner surface 11d (which is part of the wall inner surface 5d). Synergistically, the hinge devices provided in this manner extend the inner surface between segments providing a good support for the electrical cable in the area between the segments. This placement is optional but preferred to facilitate threading. The hinge devices can be offset more so that their surfaces are not on the same plane, e.g. co-extensive, with the cable lumen inner surface but, rather, form a step therewith.

FIG. 4 further shows the second hinge device 8b. Said second hinge device 8b is formed by two hinge elements 9. The hinge elements 9 of the second hinge device 8b are arranged on opposite sides of the first bending plane B, particularly at mirror symmetrical locations with respect to the first bending plane B. Further, the hinge elements 9 of the second hinge device 8b are offset from the neutral or central plane C to another side of the central plane C, i.e. to a side opposite of the side to which the hinge elements 9 of the first hinge device 8a are offset. Both hinge elements 9 of the second hinge device 8b are offset to the same side of the central plane C. The second hinge axis H2 traverses the second hinge device 8b, particularly its hinge elements 9. The location of the second hinge axis H2 is defined by the location of the second hinge device 8b. In the present embodiment, said second hinge axis H2 is parallel to the central plane C.

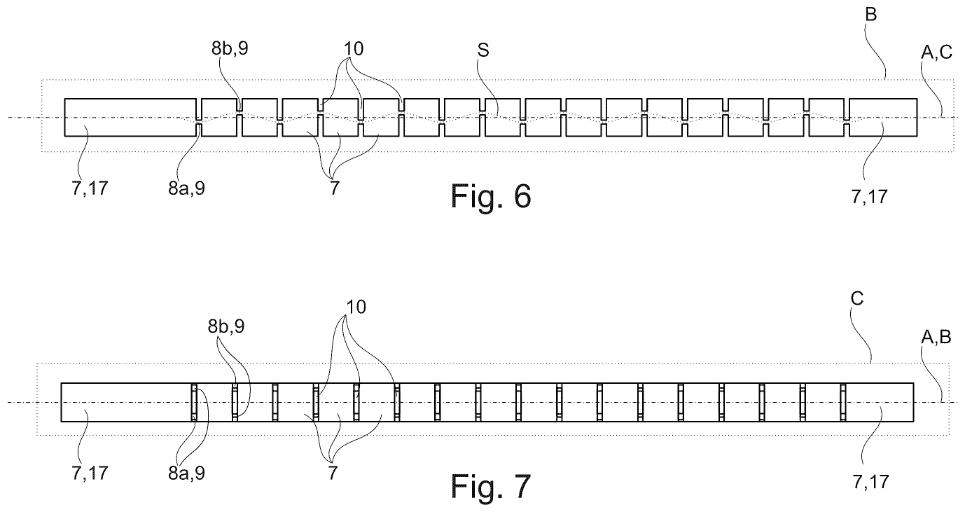

FIG. 5 shows a frontal view of a most distal segment of the segments 7 of the bending section 5. Shapes in a cross-sectional view of said most distal segment of the segments 7 and the lumen 11 formed therein are the same as those of the other segments 7, except for the following: each steering wire lumen portion 11c comprises a wire holder 16 where the respective steering wires 15 are attached in order to apply a steering force to the bending section 5. In the present embodiment, the wire holders 16 are formed as flanges or bulges or collars, e.g. clamping the steering wires 15 or providing a stop for a distal end element or portion of the steering wires 15. Further, the most distal segment of the segments 7 forms a mounting portion 17 for mounting the distal tip 6. The most proximal segment of the segments 7 also forms a mounting portion 17 for mounting the bending section 5 to the insertion tube 4. In the present embodiment, both of said mounting portions 17 are formed as a sleeve-like receptacle. Such mounting portions 17 are illustrated in FIG. 2, where the most proximal segment is shown in a perspective view or in FIG. 5 where the most distal segment is shown in a frontal view. Due to said mounting portion 17, the most distal one and/or the most proximal one of the segments 7 may be longer in a direction of the longitudinal axis L than the other segments 7, as can be seen in FIGS. 6 and 7. FIG. 7 shows a side view orthogonal to the neutral or central plane C.

FIGS. 6 and 7 illustrate the hinge devices in side views of the bending section 5. The segments 7 of the bending section 5 are alternatingly connected by first hinge devices 8a and second hinge devices 8b. I.e. a first one of the segments is connected to a distally adjacent second one of the segments 7 via a first hinge device 8a, and said second one of the segments 7 is connected to another distally adjacent third one of the segments 7 via a second hinge device 8b. Said third one of the segments 7 is in turn connected to yet another distally adjacent fourth one of the segments 7 via a first hinge device 8a, etc.

Due to the above described offset of the first hinge device 8a and the second hinge device 8b to opposite sides of the central plane C, the bending section has a spine S extending along the endoscope axis, i.e. in a proximal-distal direction, while meandering back and forth through/over the central plane C. The segments 7 extend in a rib-like manner from said meandering spine S. Further, due to the hinge devices 8a, 8b each having two hinge elements 9 on opposite sides of the first bending plane B, two such meandering spines S are provided. These spines S are indicated by a dotted line in FIGS. 2 and 6, with FIG. 6 showing a side view orthogonal to the first bending plane B. Further, due to the above described mirror-symmetrical arrangement of the two hinge elements 9 of each hinge device 8a, 8b with respect to the first bending plane B, two such meandering spines S are formed by the bending section 5 that meander substantially parallel to each other, when viewed in a side view orthogonal to the first bending plane B, as shown in FIG. 7. In the side view orthogonal to the first bending plane B shown in FIG. 6, the hinge elements 9 of the one spine S are respectively hidden behind the hinge elements 9 of the other spine 9.

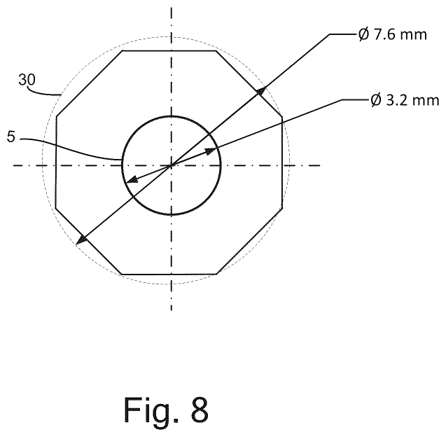

As mentioned above, a large working channel, relative to the outer diameter of the bending section, is particularly advantageous as the diameter of the bending section is reduced. The large working channel is needed, for example, to prevent backpressure during irrigation. This reduction in diameter makes it more difficult to thread wires and cables through the lumens. The wires and cables may also be reduced in gage, or diameter, to facilitate said threading, and this makes them even more susceptible to damage due to strain caused by bending. Placing the first hinge device 8a and the second hinge device 8b to opposite sides of the central plane C has the synergistic effect of allowing for a larger central lumen for the working channel tube and also supporting the cable. To facilitate comprehension of the complexities brought on by size reductions, FIG. 8 shows the cross-section of a No. 2 pencil, denoted by numeral 30, and, superimposed thereon, the cross-section of a bending section with a 3.2 mm diameter. Cameras and LEDs have typically been a limiting factor in size reductions. As the sizes of cameras and LEDs are further reduces, the limiting factor becomes the ability to assemble, economically due to the single-use nature of desirable endoscopes, the tubes, wires and cables through the insertion cord. The hinge devices and the cross-section of the bending section address these problems.

FIGS. 9 and 10 further illustrate a cable support feature first shown in FIGS. 3 and 4, which also contributes to the increased working channel lumen. FIG. 9 is a cross-sectional view of the bending section 5 taken across the central plane C. The upper portions of the wall of each segment are sectioned, as shown. As used herein, the upper portions of the wall are the portions extending from the appex of the cable lobe to the outer surface of the wall. The apex may lie in the central plane or may be slightly offset therefrom. Segments 7a and 7b form an adjacent segment pair 7ab connected by a first hinge device 8a including an upper hinge 8au. Segments 7b and 7c form an adjacent segment pair 7bc connected by a second hinge device 8b including an upper hinge (not seen in this cross-section view). Segments 7c and 7d form an adjacent segment pair 7cd connected by another first hinge device 8a including an upper hinge 8au. Segments 7d and 7e form an adjacent segment pair 7de connected by another second hinge device 8b including an upper hinge (not seen in this cross-section view). The cable lumen inner surfaces 11d of the cable lumen portions, or lobes, 11b, are shown. Co-extensive with a portion of said surfaces denoted as face 7f (shown in FIG. 10) is a face 8f of the upper hinge 8au. The bottom surface of the upper hinge 8au is denoted by numeral 21. The thickness of the upper hinge 8au is denoted as Ht and the thickness of the upper portions of the wall are denoted as Wt. As shown, the thickness Ht is greater than the thickness Wt, which allows for the provision of the face 8f adjacent the face 7f. The face 8f has a common edge with the face 7f of each segment adjacent to it.

FIGS. 11 and 12 show a variation of the present embodiment, wherein the endoscope is a four-way bending endoscope. Therein, the bending section 5 is adapted to bend in two bending planes, the first bending plane B and a second bending plane. The second bending plane preferably corresponds to the neutral plane or the central plane C. In the present variation, in addition to the first and second hinge devices 8a, 8b, the bending section 5 has third hinge devices 8c, which connect some of the segment pairs connected by the hinge devices 8a, 8b to each other. The segments 7 are alternatingly connected via first hinge devices 8a, second hinge devices 8b and third hinge devices 8c, as shown in FIG. 11.

FIG. 12 shows a cross-sectional view of the bending section in an area of one of the third hinge devices 8c. The third hinge devices 8c have two hinge elements 9 structured similar to those of the first and second hinge devices 8a, 8b, but arranged diametrically opposite to each other in the second bending plane. That is, the third hinge device 8c defines a third hinge axis H3, which extends in the first bending plane, orthogonally to the first and second hinge devices. Further, the lumen 11 forms two additional steering wire lumens 11c, which are arranged on opposing sides of the first bending plane B, particularly in or near the second bending plane, and accommodate additional steering wires 15 for controlling a bending of the bending section 5 in the second bending plane.

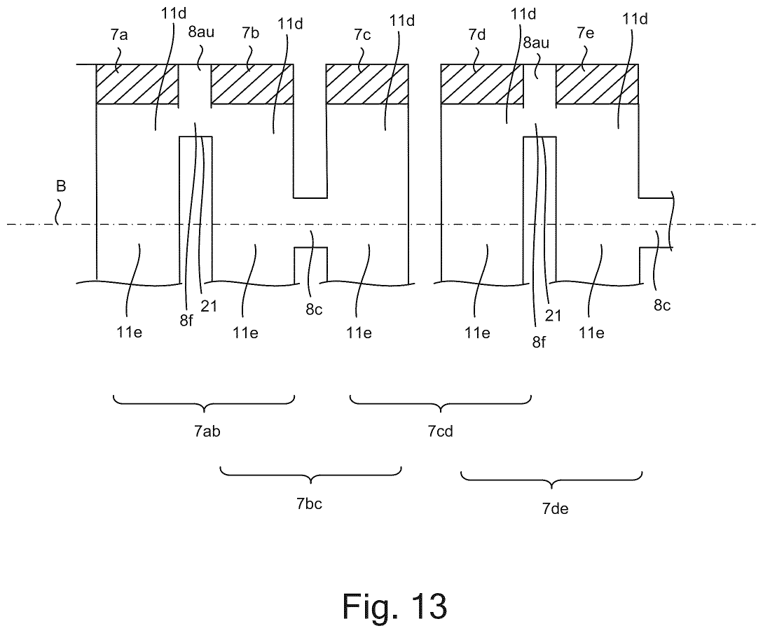

FIG. 13 further illustrate the cable support feature first shown in FIGS. 3 and 4, which also contributes to the increased working channel lumen. FIG. 13 is a cross-sectional view of the bending section 5 of FIGS. 11 and 12 taken across the central plane C. The upper portions of the wall of each segment are sectioned, as shown. Segments 7a and 7b form an adjacent segment pair 7ab connected by a first hinge device 8a including an upper hinge 8au. Segments 7b and 7c form an adjacent segment pair 7bc connected by a third hinge device 8c. Segments 7c and 7d form an adjacent segment pair 7cd connected by a second hinge device 8b. Segments 7d and 7e form an adjacent segment pair 7de connected by another second hinge device 8a. In sum, in the present variation adjacent segment pairs of segments connected by first or second hinge devices are connected by third hinge devices.

Other arrangements of the hinge devices may be provided. For example, along the axial direction, between each first and second hinge devices 8a, 8b, one third hinge device 8c (or optionally fourth hinge device) may be arranged. That is, a first one of the segments 7 may be connected to a subsequent second one of the segments 7 via the first hinge device 8a (also called a first plane-one hinge device). The second one of the segments 7 may be connected to a subsequent third one of the segments 7 via the third hinge device 8c (also called a first plane-two hinge device) for bending in the second bending plane. The third one of the segments 7 may be connected to a subsequent fourth one of the segments 7 via the second hinge device 8b (also called a second plane-one hinge device) for bending in the first bending plane. The fourth one of the segments 7 may be connected to a subsequent fifth one of the segments 7 via the another third hinge device 8c or via the fourth hinge device 8 (also called a second plane-two hinge device) for bending in the second bending plane. This order may be repeated.

Additionally, while the hinges on a side of the first bending plane B opposite the cable lobe, referred to as the lower hinges, may lie on the hinge axes H1 and H2, as described above, which allows the lumen to be closer to the outer surface of the wall, the lower hinges do not support the cable and, therefore, in another variation of the embodiments according to the first aspect, one lower hinge may be provided instead of two. The lower hinge may lie in either of the hinge axes, may lie in the central plane, or may lie between the hinge axes and the central plane.

The following items are further variations and examples of the embodiments described with reference to the figures.

1. An endoscope (1) comprising: a proximal handle or interface (2); an insertion cord (3) extending distally from the handle (2), the insertion cord comprising an insertion tube (4), a bending section (5) and a distal tip (6); a working channel tube (13); and an electrical cable (14) extending from the handle and through the bending section, the bending section comprising a one-piece part including a longitudinal axis (L), segments (7), first hinge devices (8a), and second hinge devices (8b), the first hinge devices and the second hinge devices being configured to allow bending of the bending section in a bending plane (B), each of the first hinge devices comprising a pair of hinges and a first hinge axis (H1), and each of the second hinge devices comprising a pair of hinges and a second hinge axis (H2), the first hinge axes being parallel and transversely offset from the second hinge axes, each of the segments comprising a wall (5a) with a varying thickness between an outer surface (5c) and an inner surface (5d) thereof, the inner surface forming a lumen (11) including a major lumen portion (11a), a cable lumen lobe (11b), and opposite steering wire lumen lobes (11c), the major lumen portion receiving the working channel tube therethrough, and wherein the segments are alternatingly connected to each other by the first hinge devices and the second hinge devices, forming at least one meandering spine(S), wherein the cable lumen lobe is offset from the longitudinal axis, a central plane (C) traversing the cable lumen portion and the longitudinal axis, the central plane being positioned between the first hinge axes and the second hinge axes.

2. The endoscope (1) of item 1, wherein the central axis is perpendicular to the bending plane and parallel with the first hinge axes and the second hinge axes, and wherein each of the pair of hinges of the first hinge devices and the second hinge devices includes an upper hinge (8au) adjacent to a respective one of the cable lumen lobes.

3. The endoscope (1) of item 1, wherein each of the upper hinges comprises an inner upper hinge face (8f), wherein each of the cable lumen lobes comprises a first face (7f) opposite a second face, the first face and the second face being part of the lumen (11), wherein each of the inner upper hinge faces of the first hinge devices has a common edge, and extends co-extensively, with a respective one of the first faces, and wherein each of the inner upper hinge faces of the second hinge devices has a common edge, and extends co-extensively, with a respective one of the second faces.

4. The endoscope (1) of item 1, wherein each segment comprises an upper wall portion of the wall, the upper wall portion traversed by the central plane and adjacent to the cable lumen lobe, the upper wall portion being thinner than a thickness of the upper hinges.

5. The endoscope (1) of item 1, wherein the first hinge device is offset from the central plane by at most 20% of an outer diameter of the bending section, and wherein the second hinge device is offset from the central plane by at most 20% of the outer diameter of the bending section.

6. The endoscope (1) of item 1, wherein the major lumen portion comprises arcuate portions supporting the working channel tube, whose lengths, in aggregate, are larger than 50% of a circumferential length of the working channel tube, wherein the bending section has an outer diameter smaller than 3.2 mm, and wherein an internal diameter of the working channel tube is at least 45% of the outer diameter of the bending section.

7. An endoscope (1) comprising: a proximal handle or interface (2); an insertion cord (3) extending along an endoscope axis (A) from the handle or interface (2) and configured to be inserted into a patient's body cavity, the insertion cord (3) comprising an insertion tube (4), a bending section (5) and a distal tip (6); and a steering mechanism (8) configured to swivel the distal tip (6) by bending the bending section (5) at least in a first bending plane (B); wherein the bending section (5) comprises a number of segments (7) aligned along the endoscope axis (A) and forming a cable lumen portion (11b) for accommodation of at least one electrical cable (14), said cable lumen portion (11b) being offset from the endoscope axis (A) and arranged in a central plane (C), which is substantially perpendicular to the first bending plane (B), and the segments (7) are connected by at least one first hinge device (8a) and at least one second hinge device (8b), which define the first bending plane (B) and are configured to allow bending of the bending section in the first bending plane (B), with at least one of the segments (7) being connected to a respectively adjacent one of the segments (7) by a first hinge device (8a) defining a first hinge axis (H1), which is offset from the central plane (C) in a first direction and at least another one of the segments (7) being connected to a respectively adjacent one of the segments (7) by a second hinge device (8b) defining a second hinge axis (H2), which is offset from the central plane (C) in a second direction opposite to the first direction.

8. The endoscope (1) according to item 1, wherein the first hinge device (8a) is offset from the central plane (C) by less than half a diameter of the bending section (5), preferably maximum 40%, further preferably by maximum 30%, further preferably by maximum 20% of the diameter of the bending section (5).

9. The endoscope (1) according to one of the preceding items, wherein along the endoscope axis (A), adjacent ones of the segments (7) are alternatingly connected by first hinge devices (8a) and second hinge devices (8b).

10. The endoscope (1) according to one of the preceding items, wherein the first hinge device (8a) and/or the second hinge device (8b) form film hinges and are formed in one piece of material together with the segments (7).

11. The endoscope (1) according to one of the preceding items, wherein a wall thickness of the segments (7) in the central plane (C), particularly adjacent to the cable lumen portion (11b), is thinner than a thickness of a hinge element (9) of the at least one first hinge device (8a) and/or the at least one second hinge device (8b).

12. The endoscope (1) according to item 5, wherein each of the at least one first hinge device (8a) and/or the at least one second hinge device (8b) respectively comprises two hinge elements (9), with one of said two hinge-elements (9) being arranged on one side of the first bending plane (B) and the other one of said two hinge elements (9) being arranged on the other side of the first bending plane (B), preferably such that the two hinge elements (9) are arranged mirror symmetrically with respect to the first bending plane (B).

13. The endoscope (1) according to one of the preceding items, wherein the segments (7) form a major lumen portion (11a), which has a larger cross-sectional area than the cable lumen portion (11b) and is laterally connected to the cable lumen portion (11b).

14. The endoscope (1) according to item 13, wherein in a transitional lumen wall area (12) connecting the cable lumen portion (11b) and the major lumen portion (11a), a lumen wall is formed convex.

15. The endoscope (1) according to one of the preceding items 13 and 14, wherein the major lumen portion (11a) is arranged in a cross-sectional area comprising the endoscope axis (A).

16. The endoscope (1) according to one of the preceding items, wherein the segments (7) further form at least one steering wire lumen portion (11c) for accommodation of at least one steering wire (15), said steering wire lumen portion (11c) being arranged at one side of the central plane (C).

17. The endoscope (1) according to one of the preceding items, wherein the endoscope (1) is a two-way bending endoscope, the distal tip (6) being steerable only in the first bending plane (B).

18. The endoscope (1) according to one of the preceding items, wherein the endoscope (1) is a four-way bending endoscope, the distal tip (6) being steerable in the first bending plane and in a second bending plane defined at least by third hinge devices connecting a number of the segments (7), and preferably the central plane (C) serves as the second bending plane.

19. A system comprising an endoscope (1) according to any one of the items 1 to 12 and a monitor (M) connectable to the endoscope (1).

20. An endoscope (1) comprising: a proximal handle or interface (2); an insertion cord (3) extending distally from the handle (2), the insertion cord comprising an insertion tube (4), a bending section (5) and a distal tip (6); a working channel tube (13); and an electrical cable (14) extending from the handle and through the bending section, the bending section comprising a one-piece part including a longitudinal axis (L), segments (7), first hinges (8au), second hinges, and third hinges, the first hinges, the second hinges, and the third hinges being configured to allow bending of the bending section in a bending plane (B), the first hinges comprising first hinge axis (H1), the second hinges comprising second hinge axis (H2), and the third hinges comprising third hinge axis, the first hinge axes being parallel and transversely offset from the second hinge axes, each of the segments comprising a wall (5a) with a varying thickness between an outer surface (5c) and an inner surface (5d) thereof, the inner surface forming a lumen (11) including a major lumen portion (11a), a cable lumen lobe (11b), and opposite steering wire lumen lobes (11c), the major lumen portion receiving the working channel tube therethrough, and wherein the segments are alternatingly connected to each other by the first hinges and the second hinges, forming at least one meandering spine(S), wherein the cable lumen lobe is offset from the longitudinal axis, a central plane (C) traversing the cable lumen portion and the longitudinal axis, the central plane being positioned between the first hinge axes and the second hinge axes, wherein the first hinges and the second hinges are on one side of the bending plane (B), and wherein the third hinges are on an opposite side of the bending plane (B) and longitudinally aligned with each other.

21. The endoscope (1) according to item 20, wherein the third hinges lie on the central plane C.

### LIST OF REFERENCE NUMBERS

1 Endoscope
2 Handle
3 Insertion cord
4 Insertion tube
5 Bending section
5a tubular wall
5b transverse portion of the tubular wall
6 Distal tip
7 Segments
8a, 8b First hinge devices and second hinge devices
9 Hinge elements
10 Gaps
11 lumen
11a major lumen portion
11b cable lumen portion
11c steering wire lumen portions
12 transitional areas
13 working channel tube
14 electrical cable
15 steering wires
16 wire holder
17 mounting portion
A Endoscope axis
B Bending plane
C Neutral or central plane/second bending plane
H1 First bending axis
H2 Second bending axis
H3 Third bending axis

We claim:

1. An endoscope comprising:
a proximal handle;
an insertion cord extending distally from the handle, the insertion cord comprising an insertion tube, a bending section and a distal tip;
a working channel tube; and
an electrical cable extending from the handle and through the bending section,
the bending section comprising a one-piece part including a longitudinal axis, segments, first upper hinges, and second upper hinges, the first upper hinges and the second upper hinges being configured to allow bending of the bending section in a bending plane, each of the first upper hinges comprising a first hinge axis, and each of the second upper hinges comprising a second hinge axis, the first hinge axes being parallel and transversely offset from the second hinge axes,
each of the segments comprising a wall with a varying thickness between an outer surface and an inner surface thereof, the inner surface forming a lumen including a major lumen portion, a cable lumen lobe, and opposite steering wire lumen lobes, the major lumen portion receiving the working channel tube therethrough,
wherein the segments are alternatingly connected to each other by the first upper hinges and the second upper hinges, the first upper hinges and the second upper hinges forming a meandering spine,
wherein the cable lumen lobe is offset from the longitudinal axis, a central plane traversing the cable lumen portion and the longitudinal axis, the central plane being positioned between the first hinge axes and the second hinge axes.

2. The endoscope of claim 1, wherein each of the first and second upper hinges comprises an inner upper hinge face, wherein each of the cable lumen lobes comprises a first face opposite a second face, the first face and the second face being part of the lumen, wherein each of the inner upper hinge faces of the first upper hinges has a common edge, and extends co-extensively, with a respective one of the first faces, and wherein each of the inner upper hinge faces of the second upper hinges has a common edge, and extends co-extensively, with a respective one of the second faces.

3. The endoscope of claim 1, wherein the bending section further comprises first lower hinges and second lower hinges, the first lower hinges and the second lower hinges being configured to allow bending of the bending section in the bending plane, each of the first lower hinges lying in the first hinge axis of a corresponding first upper hinge, and each of the second lower hinges lying in the second hinge axis of a corresponding second upper hinge, each of the first lower hinge and the corresponding first upper hinge forming a first hinge device, and each of the second lower hinge and the corresponding second upper hinge forming a second hinge device.

4. The endoscope of claim 3, wherein the first hinge device is offset from the central plane by at most 20% of an outer diameter of the bending section, and wherein the second hinge device is offset from the central plane by at most 20% of the outer diameter of the bending section.

5. The endoscope of claim 1,
wherein the bending section further comprises first lower hinges and second lower hinges, the first lower hinges and the second lower hinges being configured to allow bending of the bending section in the bending plane, each of the first lower hinges lying in the first hinge axis of a corresponding first upper hinge, and each of the second lower hinges lying in the second hinge axis of a corresponding second upper hinge, each of the first lower hinge and the corresponding first upper hinge forming a first hinge device, and each of the second lower hinge and the corresponding second upper hinge forming a second hinge device, and
wherein each of the first upper hinges and the second upper hinges is adjacent to a respective one of the cable lumen lobes.

6. The endoscope of claim 1, wherein each segment comprises an upper wall portion of the wall, the upper wall portion traversed by the central plane and adjacent to the cable lumen lobe, the upper wall portion being thinner than a thickness of the first or second upper hinges.

7. The endoscope of claim 1, wherein the major lumen portion comprises arcuate portions supporting the working channel tube, whose lengths, in aggregate, are larger than 50% of a circumferential length of the working channel tube, and wherein an internal diameter of the working channel tube is at least 45% of the outer diameter of the bending section.

8. The endoscope of claim 1, wherein the major lumen portion comprises arcuate portions supporting the working channel tube, whose lengths, in aggregate, are larger than 50% of a circumferential length of the working channel tube, wherein the bending section has an outer diameter smaller than 3.2 mm, and wherein an internal diameter of the working channel tube is at least 45% of the outer diameter of the bending section.

9. An endoscope comprising:
a proximal handle or interface; and
an insertion cord extending distally from the handle or interface, the insertion cord comprising an insertion tube, a bending section and a distal tip, the bending section comprising a one-piece part including a longitudinal axis, segments, a first hinge device, and a second hinge device, the first hinge device and the second hinge device being configured to allow bending of the bending section in a first bending plane; and
an electrical cable extending from the handle and through the bending section,
wherein the bending section comprises a cable lumen portion offset from the longitudinal axis, a central plane traversing the cable lumen portion and the longitudinal axis, the electrical cable extending through the cable lumen portion,
wherein one of the segments is connected to a respectively adjacent one of the segments by the first hinge device, a first hinge axis traversing the first hinge device and being offset from the central plane in a first direction,
wherein another one of the segments is connected to a respectively adjacent another one of the segments by the second hinge device, a second hinge axis traversing the second hinge device and being offset from the central plane in a second direction opposite to the first direction, and
wherein the first hinge axis does not traverse the second hinge device and the second hinge axis does not traverse the first hinge device.

10. The endoscope of claim 9, wherein the central axis is perpendicular to the first bending plane and parallel with the first hinge axis and the second hinge axis.

11. The endoscope of claim 9, wherein the first hinge device is offset from the central plane by at most 20% of a diameter of the bending section.

12. The endoscope of claim 9, wherein each of the first hinge device and the second hinge device respectively comprises two hinge elements, with one of said two hinge elements being arranged on one side of the first bending plane and the other of said two hinge elements being arranged on the other side of the first bending plane.

13. The endoscope of claim 12, wherein the two hinge elements of the first hinge device and of the second hinge device are arranged mirror symmetrically with respect to the first bending plane.

14. The endoscope of claim 9, wherein each of the first hinge device and the second hinge device respectively comprises two hinge elements, with one of said two hinge elements being arranged on one side of the first bending plane and the other of said two hinge elements being arranged on the other side of the first bending plane, wherein a wall thickness of the segments, in the central plane and adjacent to the cable lumen portion, is thinner than a thickness of a hinge element of the first hinge device.

15. The endoscope of claim 9, wherein each of the first hinge device and the second hinge device respectively comprises two hinge elements, with one of said two hinge elements being arranged on one side of the first bending plane and the other of said two hinge elements being arranged on the other side of the first bending plane, and wherein the two hinge elements of the first hinge device and/or the second hinge device are arranged mirror symmetrically with respect to the first bending plane.

16. The endoscope of claim 9, wherein the segments form a major lumen portion, which has a larger cross-sectional area than the cable lumen portion and is connected to the cable lumen portion.

17. The endoscope of claim 9, wherein the segments further form at least one steering wire lumen portion for accommodation of at least one steering wire, said steering wire lumen portion being arranged at one side of the central plane and connected to the major lumen portion and the cable lumen portion.

18. The endoscope of claim 9, wherein the endoscope is a four-way bending endoscope, the distal tip being steerable in the first bending plane and in a second bending plane defined at least by third hinge devices connecting some of the segments.

19. The endoscope of claim 18, wherein the third hinge devices comprise hinge elements traversed by the second bending plane.

20. A system comprising the endoscope of claim 9 and a monitor (M) connectable to the endoscope.
